(19) United States
(12) Patent Application Publication
(10) Pub. No.: US 2006/0200205 A1
(43) Pub. Date: Sep. 7, 2006

(54) Systems and methods for treating a patient with multiple stimulation therapies

(76) Inventor: Matthew I. Haller, Valley Village, CA (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/069,251

(22) Filed: Mar. 1, 2005

(51) Int. Cl. A61N 1/372; A61N 1/36

(52) U.S. Cl. 607/41

(57) ABSTRACT

Exemplary systems for treating a patient with multiple stimulation therapies include a stimulator configured to automatically apply two or more stimulation signals to a stimulation site via a single channel. Each of the stimulation signals is defined by a distinct set of stimulation parameters. Exemplary methods of treating a patient with multiple stimulation therapies include defining two or more stimulation signals with two or more sets of stimulation parameters and applying the stimulation signals via a single channel to a stimulation site with a stimulator.

### BACKGROUND

Urinary incontinence is a clinical condition characterized by failure to hold urine in the bladder under normal conditions of pressure and filling. The most common forms of the disorder can arise from either a failure of muscles around the bladder neck and urethra to maintain closure of the urinary outlet (so-called stress incontinence) or from abnormally heightened commands from the spinal cord to the bladder that produce unanticipated bladder contractions (so-called urge incontinence). Many patients exhibit a grouping of symptoms suggesting that these disorders may occur simultaneously in the same individual (so-called mixed incontinence). Some studies have shown that mixed incontinence is five times more common than either stress or urge incontinence.

Electrical stimulation in the region of the pelvic floor can decrease the severity of incontinence. This decrease in severity is believed to be attained by changing the reflex thresholds of the bladder muscles responsible for bladder emptying; strengthening the muscles that maintain closure on the bladder outlet; and changing the state of the neural pathways, musculature, and/or bladder during and beyond the period of stimulus application.

Several external and implantable approaches have been used to stimulate the nerves supplying the bladder and pelvic region in order to treat patients suffering from urinary incontinence. However, the stimulation parameters used to treat stress and urge incontinence differ greatly. For example, stress incontinence is often treated by applying relatively high frequency, low duration electrical pulses in order to strengthen periurethral muscles. Urge incontinence, on the other hand, is often treated by applying relatively low frequency, high duration electrical pulses in order to diminish or inhibit the heightened reflexes of bladder muscles. Thus, it is currently difficult to treat a patient suffering from both stress and urge incontinence.

### SUMMARY

Exemplary systems for treating a patient with multiple stimulation therapies include a stimulator configured to automatically apply two or more stimulation signals to a stimulation site via a single channel. Each of the stimulation signals is defined by a distinct set of stimulation parameters.

Exemplary methods of treating a patient with multiple stimulation therapies include defining two or more stimulation signals with two or more sets of stimulation parameters and applying the stimulation signals via a single channel to a stimulation site with a stimulator.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1 illustrates an exemplary implantable stimulator according to principles described herein.

FIG. 2 illustrates an exemplary stimulator that is coupled to a lead with one or more electrodes according to principles described herein.

FIG. 3 is a block diagram illustrating some of the components of an exemplary implantable pulse generator that is coupled to a number of electrodes according to principles described herein.

FIG. 4 illustrates two exemplary stimulation signals having electrical stimulation pulses that may be applied to a stimulation site according to principles described herein.

FIG. 5 illustrates that the stimulator may alternate between a first stimulation signal and a second stimulation signal according to principles described herein.

FIG. 6 is a flow chart illustrating an exemplary method of applying multiple stimulation therapies to a single stimulation site with a single stimulator according to principles described herein.

Throughout the drawings, identical reference numbers designate similar, but not necessarily identical, elements.

### DETAILED DESCRIPTION

Systems and methods for treating a patient with multiple stimulation therapies are described herein. An implantable stimulator is configured to automatically apply two or more stimulation signals to a stimulation site via one or more electrodes. Each of the stimulation signals is defined by a distinct set of stimulation parameters. The patient, therefore, does not have to manually change the stimulation parameters every time a new stimulation therapy is to be applied to the stimulation site.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment. Furthermore, all patents, publications, and other documents listed or discussed herein are hereby incorporated by reference in their respective entireties.

As used herein and in the appended claims, unless otherwise specifically denoted, the term “stimulation site” will be used to refer to any nerve, muscle, organ, or other tissue within a patient that is stimulated by an implantable stimulator. For example, in the case of urinary incontinence, the stimulation site may be, but is not limited to, any nerve or muscle in the pelvic floor. Nerves in the pelvic floor region that may be targeted for stimulation include, but are not limited to, the pudendal nerve, pelvic nerve, and the clitoral branches of the pudendal nerve.

The pudendal nerve, its branches, and all somatic nerves emanating from the sacral nerve roots may be stimulated to treat dysfunctions of perineal structures, such as urinary incontinence, urgency, frequency, and/or pain or similar conditions of the bowls. For instance, stimulation of the urethral branch of the pudendal nerve may be used to inhibit defecation, thereby treating fecal incontinence. Additionally or alternatively, stimulation of the inferior rectal branch of the pudendal nerve, which innervates the external anal sphincter, may also inhibit defecation, thereby treating fecal incontinence. Stimulation of other somatic nerves innervating the rectum and/or colon may treat constipation, fecal retention, and/or colorectal hypomotility. Stimulation of one or more other pudendal nerve branches (e.g., the dorsal nerve of the clitoris or penis) may be used as a treatment of, e.g., urinary urge incontinence and/or detrusor hyperreflexia. Stimulation of nerves innervating the urethra and/or detrusor muscle may treat urinary retention, while stimulation of nerves innervating the internal and/or external urethral sphincter or their intramuscular branches may treat urinary stress incontinence. Stimulation of nerve(s) innervating the clitoris and/or vagina may treat vaginismus, dyspareunia, anorgasmia, or other female sexual dysfunction.

It will be recognized, therefore, that in addition to urinary incontinence, the stimulator described herein may be used to treat a variety of disorders, injuries, and/or pain associated with any part of the body of a patient. For example, the stimulator described herein may be used to treat any of the medical conditions described in U.S. Pat. No. 6,405,079, which patent is incorporated herein by reference in its entirety. Although the examples presented herein describe stimulation therapies for urinary incontinence, it will be recognized that the systems and methods described herein may be applied to the stimulation of any stimulation site to which it is desired to apply more than one type of stimulation therapy.

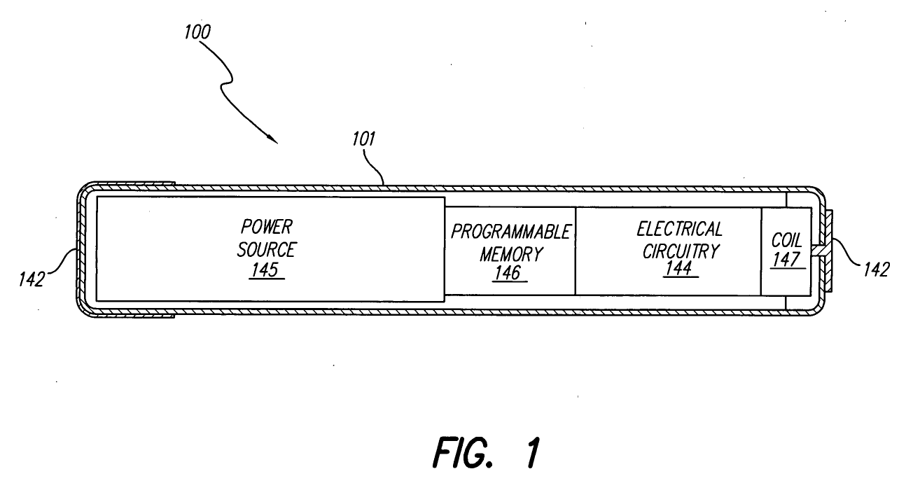

FIG. 1 illustrates an exemplary implantable stimulator (100). The stimulator (100) of FIG. 1 is a BION® microstimulator (Advanced Bionics® Corporation, Valencia, Calif.) for illustrative purposes. Various details associated with the manufacture, operation, and use of BION implantable microstimulators are disclosed in U.S. Pat. Nos. 5,193,539; 5,193,540; 5,312,439; 6,185,452; 6,164,284; 6,208,894; and 6,051,017. All of these listed patents are incorporated herein by reference in their respective entireties.

It will be recognized that the stimulator (100) of FIG. 1 may alternatively include an implantable pulse generator (IPG) coupled to a lead of electrodes, a spinal cord stimulator (SCS), a cochlear implant, a deep brain stimulator, a drug pump, a micro-drug pump or any other type of implantable stimulator configured to deliver electrical and/or drug stimulation. Exemplary IPGs suitable for use as described herein include, but are not necessarily limited to, those disclosed in U.S. Pat. Nos. 6,381,496, 6,553,263; and 6,760,626. Exemplary spinal cord stimulators suitable for use as described herein include, but are not necessarily limited to, those disclosed in U.S. Pat. Nos. 5,501,703; 6,487,446; and 6,516,227. Exemplary cochlear implants suitable for use as described herein include, but are not necessarily limited to, those disclosed in U.S. Pat. Nos. 6,219,580; 6,272,382; and 6,308,101. Exemplary deep brain stimulators suitable for use as described herein include, but are not necessarily limited to, those disclosed in U.S. Pat. Nos. 5,938,688; 6,016,449; and 6,539,263. Exemplary drug pumps suitable for use as described herein include, but are not necessarily limited to, those disclosed in U.S. Pat. Nos. 4,562,751; 4,678,408; 4,685,903; 5,080,653; 5,097,122; 6,740,072; and 6,770,067. Exemplary micro-drug pumps suitable for use as described herein include, but are not necessarily limited to, those disclosed in U.S. Pat. Nos. 5,234,692; 5,234,693; 5,728,396; 6,368,315; 6,666,845; and 6,620,151. All of these listed patents are incorporated herein by reference in their respective entireties.

As used herein and in the appended claims, unless otherwise specifically denoted, the terms “stimulator” and “microstimulator” will be used interchangeably to refer to any implantable stimulator that may be implanted within the patient and configured to provide electrical and/or other types of stimulation to a nerve, muscle, organ, and/or other tissue within a patient. The other types of stimulation may include, for example, drug stimulation wherein one or more stimulating drugs are infused into the nerve, muscle, organ, and/or other tissue.

As illustrated in FIG. 1, the stimulator (100) may include a number of components. It will be recognized that the stimulator (100) may include additional and/or different components as best serves a particular application. A power source (145) is configured to output voltage used to supply the various components within the stimulator (100) with power. The power source (145) may be a primary battery, a rechargeable battery, a capacitor, an electrical charge storage device, or any other suitable power source. In some alternative embodiments, the stimulator (100) does not include an internal power source (145) and is instead powered transcutaneously via an RF field by an external power supply. Alternatively, the stimulator (100) may include one or more components configured to receive power from another medical device that is implanted within the patient. A coil (147) is configured to receive and/or emit a magnetic field (also referred to as a radio frequency (RF) field) that is used to communicate with and/or receive power from one or more external devices (not shown) that are external to the body of the patient. Such communication and/or power transfer may include, but is not limited to, transcutaneously receiving data from the external device, transmitting data to the external device, and/or receiving power used to recharge the power source (145).

The stimulator (100) may also include electrical circuitry (144) configured to produce a stimulation signal that is delivered to a nerve, muscle, tissue, and/or other stimulation site via one or more electrodes (142). The electrodes (142) may be located at either end of the stimulator (100), as shown in FIG. 1. Alternatively, the electrodes (142) may be disposed in other locations, as will be described in more detail below.

The stimulation signal may include a number of electrical stimulation pulses and may be delivered to the stimulation site via one or more channels. As used herein and in the appended claims, unless otherwise specifically denoted, the term “channel” will be used to refer to a pathway used to deliver one or more stimulation signals to a stimulation site. Some types of stimulators (100) include multiple channels. For example, some stimulators (100) have up to sixteen or more channels. Other types of stimulators (100), such as the BION microstimulator, may include only one channel.

In some embodiments, the stimulator (100) may be configured to produce monopolar electrical stimulation. The stimulator (100) may alternatively or additionally be configured to produce bipolar electrical stimulation. The electrical circuitry (144) may include one or more processors configured to decode stimulation parameters and generate the stimulation pulses. The electrical circuitry (144) may also include additional circuitry such as capacitors, integrated circuits, resistors, coils, and the like configured to perform a variety of functions as best serves a particular application.

The stimulator (100) may also include a programmable memory unit (146) for storing one or more sets of data and/or stimulation parameters. The stimulation parameters may include, but are not limited to, electrical stimulation parameters and drug stimulation parameters. The programmable memory (146) allows a patient, clinician, or other user of the stimulator (100) to adjust the stimulation parameters such that the electrical stimulation and/or drug stimulation are at levels that are safe and efficacious for a particular patient. The programmable memory (146) may be any type of memory unit including, but not limited to, random access memory (RAM), static RAM (SRAM), a hard drive, or the like. The stimulation parameters will be described in more detail below.

As shown in FIG. 1, the components included in the stimulator (100) are housed within a capsule (101). The capsule (101) may be a thin, elongated cylinder or any other shape as best serves a particular application. The shape of the capsule (101) may be determined by the structure of the desired target, the surrounding area, and/or the method of implantation. The diameter of the capsule (101) may be less than 5 millimeters (mm) and the length of the capsule (101) may be less than 40 mm in some examples. However, it will be recognized that the diameter, width, and/or length of the capsule (101) may be any size.

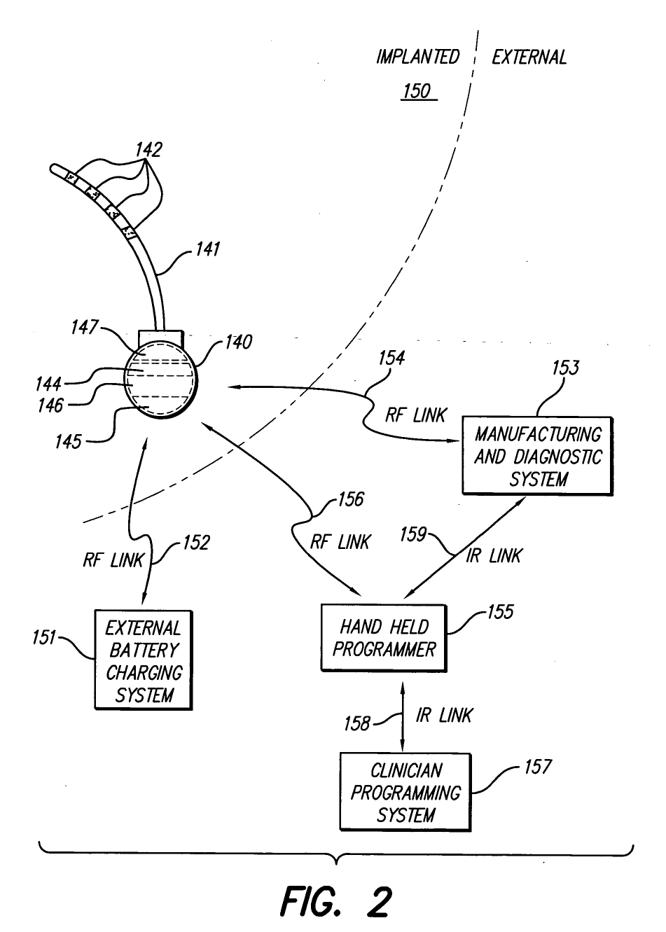

FIG. 2 illustrates an exemplary stimulator (100) that is coupled to one or more leads (141) of one or more electrodes (142). The stimulator (100) shown in FIG. 2 is an IPG configured to generate stimulation that is delivered to a stimulation site within a patient (150) via the electrodes (142). As shown in FIG. 2, the stimulator (100) includes the electrical circuitry (144), the coil (147), the power source (145), and the programmable memory (146) as described in connection with FIG. 1.

The lead (141) includes any number of electrodes (142) as best serves a particular application. For example, the lead (141) may include between two and sixteen electrodes (142). The lead (141) may be thin (e.g., less than 3 millimeters in diameter) such that the lead (141) may be positioned near a stimulation site.

As shown in FIG. 2, the stimulator (100) may communicate with a number of external devices. For example, an external battery charging system (EBCS)

(151) may provide power used to recharge the power source (145) via an RF link (152). External devices including, but not limited to, a hand held programmer (HHP) (155), clinician programming system (CPS) (157), and/or a manufacturing and diagnostic system (MDS) (153) may be configured to activate, deactivate, program, and test the stimulator (100) via one or more RF links (154, 156). The CPS (157) may communicate with the HHP (155) via an infrared (IR) link (158) or via any other suitable communication link. Likewise, the MDS (153) may communicate with the HHP (155) via an IR link (159) or via any other suitable communication link. The HHP (155), MDS (153), CPS (157), and EBCS (151) are merely illustrative of the many different external devices that may be used in connection with the stimulator (100). Furthermore, it will be recognized that the functions performed by the HHP (155), MDS (153), CPS (157), and EBCS (151) may be performed by a single external device. One or more of the external devices (153, 155, 157) may be embedded in a seat cushion, mattress cover, pillow, garment, belt, strap, pouch, or the like.

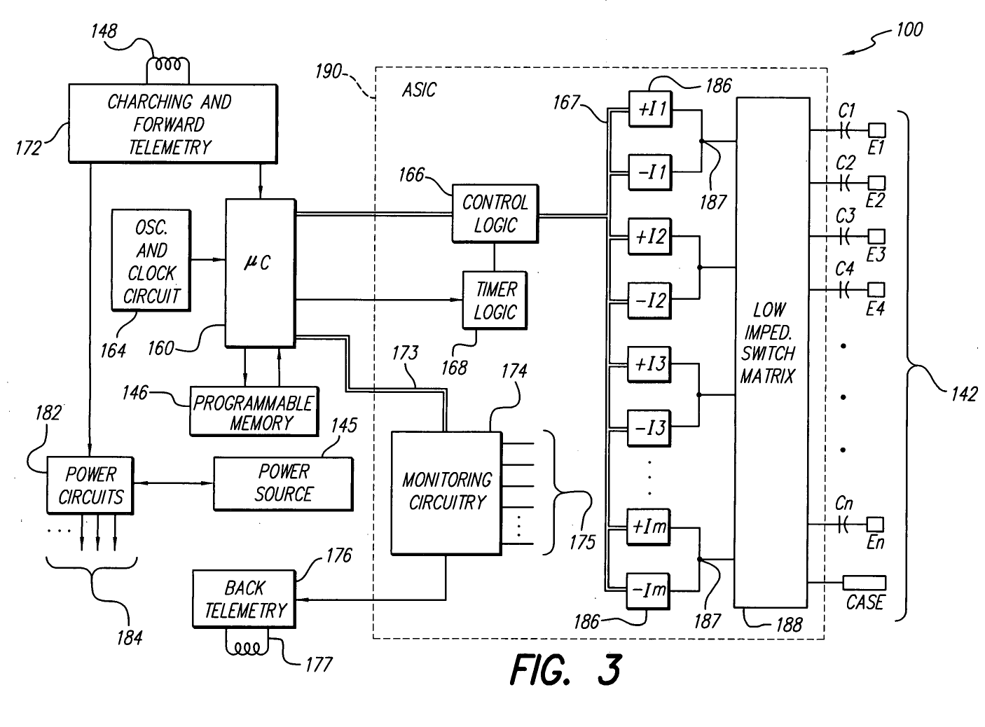

FIG. 3 is a block diagram illustrating some of the components of an IPG (130). As seen in FIG. 3, a microcontroller (μC) (160) is connected to the programmable memory (146). The microcontroller (160) typically comprises a microprocessor and associated logic circuitry, which in combination with control logic circuits (166), timer logic (168), and an oscillator and clock circuit (164), generate the necessary control and status signals which allow the microcontroller (160) to control the operation of the IPG (130) in accordance with selected stimulation parameters. The microcontroller (160) may alternatively include a state machine. The stimulation parameters may be programmed by the patient, a clinician, or by some other person or electronic device and transmitted to the programmable memory (146) via the coil (147) and charging and forward telemetry circuitry (172).

The microcontroller (160) is further coupled to monitoring circuitry (174) via bus (173). The monitoring circuitry (174) monitors the status of various nodes or other points (175) throughout the IPG (130). For example, the monitoring circuitry (174) may monitor power supply voltages, current values, temperature, the impedance of electrodes attached to the various electrodes E1 . . . En, and the like. Informational data sensed by the monitoring circuitry (174) may be sent to an external device (not shown) using back telemetry circuitry (176) and a transmission coil (177).

The power source (145) provides the operating power for the IPG (130) by providing an unregulated voltage to power circuits (182). The power circuits (182), in turn, generate various voltages (184) as needed by the various circuits located within the IPG (130).

The IPG (130) may include a number of stimulus current generators (186) configured to generate stimulus current according to the stimulation parameters in the programmable memory (146). The current generators (186) are coupled to the electrodes (142) using a switching matrix (188). As shown in FIG. 3, there may be n electrodes (142). The stimulus current generated by the stimulus current generators (186) may be delivered to the desired stimulation site via one or more of the n electrodes (142). As will be explained in more detail below, the frequency, amplitude, duration, timing, duty cycle, and pulse width of the electrical stimulus pulses delivered to the stimulation site via one or more of the n electrodes (142) may be programmed as best serves a particular application. Furthermore, the configuration of each electrode (142) may be programmed as best serves a particular application.

As shown in FIG. 3, much of the circuitry included within the IPG (130) may be realized on a single application specific integrated circuit (ASIC) (190). This allows the overall size of the IPG (130) to be relatively small, and readily housed within a suitable hermetically-sealed case.

As mentioned, the stimulator (100) may be configured to stimulate a stimulation site according to one or more sets of stimulation parameters. The stimulation parameters control the frequency, amplitude, duration, timing, pulse width, and/or duty cycle of the electrical stimulation pulses that are delivered to the stimulation site. The stimulation parameters may also control the configuration of the electrodes (142) (i.e., which electrodes are used to apply the stimulation, the polarity of the electrodes, etc.). The stimulation parameters may also control any other characteristic of the electrical stimulation pulses that are delivered to the stimulation site.

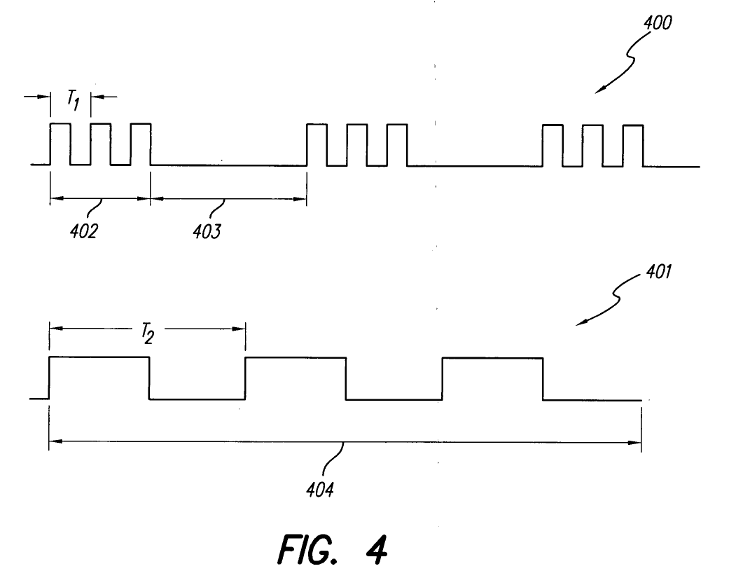

FIG. 4 illustrates two exemplary electrical stimulation signals having electrical stimulation pulses that may be used to stimulate a stimulation site. The stimulation signals shown in FIG. 4 are merely illustrative of the many different stimulation signals that may be used to stimulate a stimulation site. As shown in FIG. 4, the first stimulation signal (400) includes a number of pulses having a first frequency (1/T₁) and a duty cycle of 50 percent. The first stimulation signal (400) may be applied in a “burst-on/burst-off” mode. In other words, a train of pulses is applied during a first time period (402) (burst-on mode) followed by a second time period (403) of silence (burst-off mode). The second stimulation signal (401) includes a number of pulses having a second frequency (1/T₂) and a duty cycle of 50 percent. The second stimulation signal (401) includes a number of pulses that are applied to the stimulation site during a time period (404). The characteristics of the first and second stimulation signals (400, 401) are determined by the stimulation parameters. Hence, FIG. 4 illustrates that stimulation signals may have different stimulation parameters.

As mentioned, the stimulation parameters may be adjusted as best serves a particular stimulation therapy. For example, stress incontinence is often treated by applying a stimulation signal having relatively high frequency pulses for a relatively short period of time. For example, the pulses may be 50-100 Hz and may be applied to the stimulation site for up to 30 minutes per day. Urge incontinence, on the other hand, is often treated with a stimulation signal having relatively low frequency pulses for a relatively long period of time. For example, the pulses may be 10-20 Hz and may be applied to the stimulation site for a few hours every day. It will be recognized that these frequencies and time durations are merely illustrative of the many different frequencies and time durations that may be used to treat stress and urge incontinence.

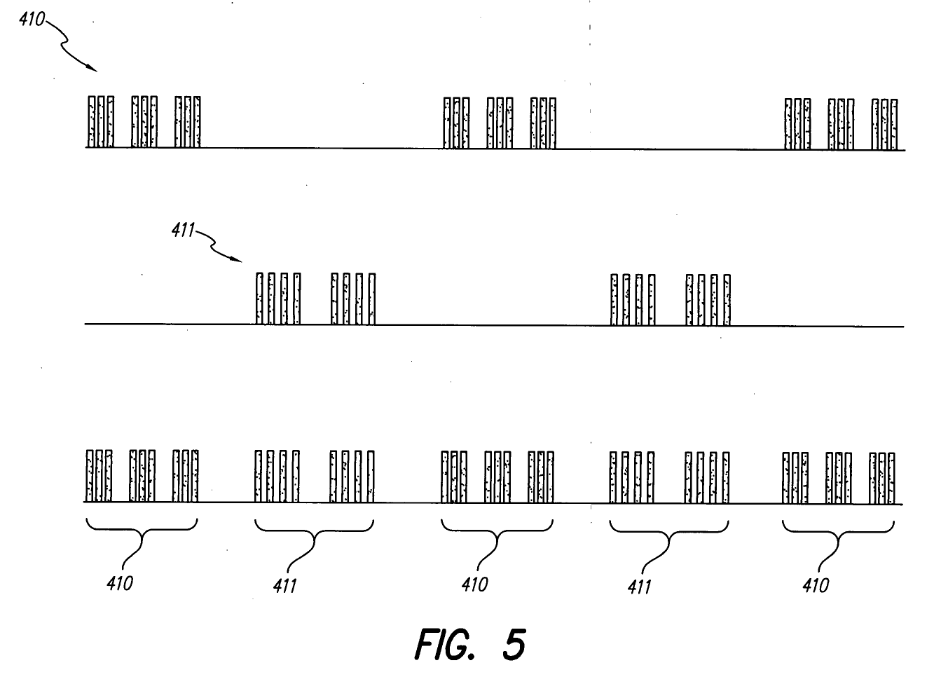

In some embodiments, the stimulator (100; FIG. 1) is configured to stimulate a single stimulation site with multiple stimulation therapies. In other words, the stimulator (100; FIG. 1) is configured to stimulate the stimulation site according to multiple sets of stimulation parameters. For example, as shown in FIG. 5, the stimulator (100; FIG. 1) may alternate between a first stimulation signal (410) and a second stimulation signal (411). The first stimulation signal (410) corresponds to a first stimulation therapy and is determined by a first set of stimulation parameters. The second stimulation signal (411) corresponds to a second stimulation therapy and is determined by a second set of stimulation parameters. In some embodiments, the first stimulation signal (410) is configured to treat stress incontinence and the second stimulation signal (411) is configured to treat urge incontinence. The bottom-most graph of FIG. 5 shows that the stimulator (100; FIG. 1) may apply the first and second stimulation signals (410, 411) in an alternating manner. Although FIG. 5 shows that two stimulation signals are applied to a single stimulation site, it will be recognized that the stimulator (100; FIG. 1) may be configured to apply any number of stimulation signals to a single stimulation site in any order.

Furthermore, it will be recognized that the stimulator (100; FIG. 1) may be configured to apply the stimulation signals according to a number of different stimulation patterns. For example, the stimulator (100; FIG. 1) may apply the first and second stimulation signals (410, 411) in an alternating pattern as described in connection with FIG. 5. Alternatively, the stimulator may be configured to apply the first stimulation signal (410) a pre-determined number of times before switching to the second stimulation signal (411).

Hence, a stimulator (100; FIG. 1) that is configured to automatically apply multiple stimulation signals allows a single stimulation site to be treated with multiple stimulation therapies. A patient, therefore, does not have to manually change the stimulation parameters every time a new stimulation therapy is to be applied to the stimulation site. In the case of mixed urinary incontinence, for example, the patient does not have to manually change stimulation parameters to treat stress and urge incontinence. Rather, the stimulator (100; FIG. 1) is configured to automatically deliver different stimulation signals a single stimulation site to treat both stress and urge incontinence. It will be recognized that the methods and systems described herein may also or alternatively be used to treat many types of medical conditions and/or disorders other than mixed urinary incontinence. It will also be recognized that the stimulator (100; FIG. 1) may be configured to apply any number of stimulation signals therapies in any order according to any stimulation pattern.

In some embodiments, the multiple stimulation signals are delivered to the stimulation site via a single channel within the stimulator (100; FIG. 1). For example, a single channel stimulator, such as the BION microstimulator, may be configured to deliver the first and second stimulation signals (410, 411) to a stimulation site in the manner described in connection with FIG. 5. The use of a single channel stimulator is advantageous in many stimulation therapy applications where it is desirable for the stimulator to be minimally intrusive. For example, a single channel stimulator that can be coupled to the pudendal nerve is desirable in many treatments of urinary incontinence. However, the methods and systems described herein are not limited to a single channel stimulator. In some embodiments, a multi-channel stimulator may be configured to deliver the first and second stimulation signals (410, 411) of FIG. 5 to a stimulation site via a single channel either as a part of a multi-channel stimulation regime or as a single channel stimulation regime.

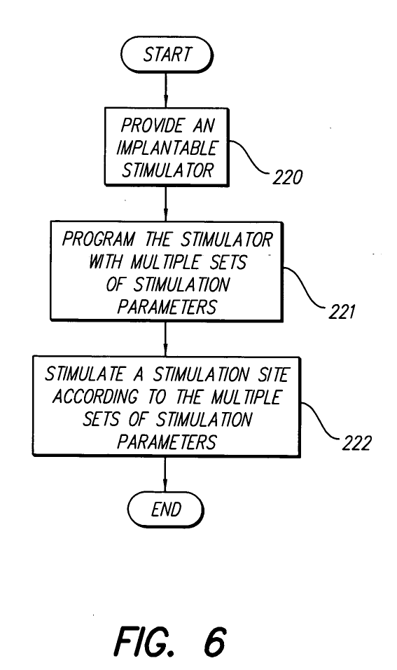

FIG. 6 is a flow chart illustrating an exemplary method of applying multiple stimulation therapies to a single stimulation site with a single stimulator (100; FIG. 1). The steps shown in FIG. 6 are merely illustrative and may be modified and/or added to as best serves a particular application. An implantable stimulator (100; FIG. 1) is first provided (step 220). The stimulator (100; FIG. 1) is then programmed with multiple sets of stimulation parameters (step 221). The stimulator (100; FIG. 1) then stimulates the stimulation site according to the multiple sets of stimulation parameters (step 222).

The preceding description has been presented only to illustrate and describe embodiments of the invention. It is not intended to be exhaustive or to limit the invention to any precise form disclosed. Many modifications and variations are possible in light of the above teaching.

What is claimed is:

1. A system for treating a stimulation site within a patient with multiple stimulation therapies, said system comprising:
a stimulator configured to automatically apply two or more stimulation signals to said stimulation site via a single channel;
wherein each of said stimulation signals is defined by a distinct set of stimulation parameters.

2. The system of claim 1, wherein said stimulation signals comprise:
a first stimulation signal configured to treat stress incontinence; and
a second stimulation signal configured to treat urge incontinence.

3. The system of claim 1, wherein each of said sets of said stimulation parameters comprises at least one or more of a frequency, amplitude, duration, timing, duty cycle, pulse width, and configuration of said channel corresponding to one of said stimulation signals.

4. The system of claim 1, wherein said stimulator comprises a programmable memory unit for storing said sets of stimulation parameters.

5. The system of claim 1, wherein said stimulator comprises at least one or more of a microstimulator and an implantable pulse generator.

6. The system of claim 1, wherein said stimulation site is located in a pelvic region of said patient.

7. The system of claim 1, wherein said stimulator comprises a single channel stimulator.

8. The system of claim 1, wherein said stimulator comprises a multi-channel stimulator.

9. A method of treating a stimulation site within a patient with multiple stimulation therapies, said method comprising:
defining two or more stimulation signals with two or more sets of stimulation parameters; and
applying said stimulation signals via a single channel to said stimulation site with a stimulator.

10. The method of claim 9, wherein said stimulation signals comprise:
a first stimulation signal configured to treat stress incontinence; and
a second stimulation signal configured to treat urge incontinence.

11. The method of claim 9, wherein each of said sets of said stimulation parameters comprises at least one or more of a frequency, amplitude, duration, timing, duty cycle, pulse width, and configuration of said channel corresponding to one of said stimulation signals.

12. The method of claim 9, further comprising storing said sets of stimulation parameters in a programmable memory unit.

13. The method of claim 9, wherein said stimulator comprises at least one or more of a microstimulator and an implantable pulse generator.

14. The method of claim 9, wherein said stimulation site is located in a pelvic region of said patient.

15. The method of claim 9, wherein said step of applying said stimulation signals via said single channel to said stimulation site comprises applying said stimulation signals in an alternating pattern.

16. A system for treating a stimulation site within a patient with multiple stimulation therapies, said system comprising:
means for defining two or more stimulation signals with two or more sets of stimulation parameters; and
means for applying said stimulation signals via a single channel to said stimulation site.

17. The system of claim 16, wherein said stimulation signals comprise:
a first stimulation signal configured to treat stress incontinence; and
a second stimulation signal configured to treat urge incontinence.

18. The system of claim 16, wherein each of said sets of said stimulation parameters comprises at least one or more of a frequency, amplitude, duration, timing, duty cycle, pulse width, and configuration of said channel corresponding to one of said stimulation signals.

19. The system of claim 16, further comprising means for storing said sets of stimulation parameters in a programmable memory unit.

20. The system of claim 16, wherein said stimulation site is located in a pelvic region of said patient.
